(19) United States
(12) Patent Application Publication
(10) Pub. No.: US 2015/0096140 A1
(43) Pub. Date: Apr. 9, 2015

(54) ANTI-FOG WIPE CONSTRUCTION

(71) Applicant: John Temple, Chelsea, MI (US)

(72) Inventor: John Temple, Chelsea, MI (US)

(21) Appl. No.: 14/504,473

(22) Filed: Oct. 2, 2014

Related U.S. Application Data
(60) Provisional application No. 61/886,408, filed on Oct. 3, 2013.

(51) Int. Cl. A61B 1/12

(52) U.S. Cl. CPC A61B 1/122; A61B 1/127; A61B 2019/343
USPC 15/210.1

(57) ABSTRACT

An improved wipe for a laparoscope or other optical device subject to fogging comprises a top outer layer of microfiber cloth, a bottom outer layer, and absorbent material disposed between the top and bottom outer layers. The bottom layer may include an adhesive layer exposed by a release layer. The absorbent material may be a sponge or foam pad, including an open-cell foam pad. Alternatively, the absorbent material may include a plurality of absorbent fabric layers, with at least the outermost layer being a microfiber cloth layer. A sponge or foam tape may be disposed between the absorbent fabric layers and the bottom outer layer. The wipe may be a square shape with dimensions on the order of 2 to 4 inches on a side and a thickness in the range of ⅛ to ½ inches. The article may further include a radiopaque thread.

### REFERENCE TO RELATED APPLICATION

This application claims priority from U.S. Provisional Patent Application Ser. No. 61/886,408, filed Oct. 3, 2013, the entire content of which is incorporated herein by reference.

### FIELD OF THE INVENTION

This invention relates generally to cleaning cloths and, in particular, to an anti-fog wipe used with endoscope, laparoscopes and other viewing instruments.

### BACKGROUND OF THE INVENTION

Currently in conjunction with minimally invasive surgical procedures, anti-fogging agents are applied to hydrophilic sponges used to wipe the ends of laparoscopes and other such instruments. Sponges by their nature are slightly abrasive, causing very fine micro-scratches to eventually occur on the scopes lens. Indeed, an entire industry has developed to repair these scopes.

Microfiber cloths are made from super-fine synthetic fibers using polyesters, polyamides (e.g., nylon, Kevlar, Nomex, trogamide), or a combination thereof. Microfiber cloths are used to make optical lens wipes because they have soft and non-abrasive surfaces. However a single layer of micro-cloth is not absorbent enough to be used for surgical scope cleaning applications. In fact, single layers tend to be non-absorbent. In addition, because the micro-cloth is relatively thin, it does not supply enough impact absorption to prevent damage to the scope when it is being wiped against a hard surface, which is also the case in general use.

### SUMMARY OF THE INVENTION

This invention is directed to an improved wipe for a laparoscope or other optical device subject to fogging, comprising. The article comprises a top outer layer of microfiber cloth chosen so as not to scratch the optics of the device, a bottom outer layer, and absorbent material disposed between the top and bottom outer layers. The bottom layer may include an adhesive layer exposed by a release layer.

The absorbent material may be a sponge or foam pad, including an open-cell foam pad. Alternatively, the absorbent material may include a plurality of absorbent fabric layers, with at least the outermost layer being a microfiber cloth layer. One or more of the absorbent fabric layers may be made of terrycloth. A sponge or foam layer may be disposed between the absorbent fabric layers and the bottom outer layer. If one or more absorbent fabric layers are used, the sponge or foam layer may be a closed-cell foam pad.

The wipe may be a square shape with dimensions on the order of 2 to 4 inches on a side and a thickness in the range of ⅛ to ½ inches. The article may further include a radiopaque thread.

### BRIEF DESCRIPTION OF THE DRAWINGS

FIG. 1A is a top view of a first embodiment of the invention;

FIG. 1B is a side view of the embodiment of FIG. 1A;

FIG. 1C is a bottom view of the embodiment of FIG. 1A;

FIG. 1D is a cross section taken along A-A designated in FIG. 1A;

FIG. 2A is a top view of a first embodiment of the invention;

FIG. 2B is a side view of the embodiment of FIG. 2A;

FIG. 2C is a bottom view of the embodiment of FIG. 2A; and

FIG. 2D is a cross section taken along A-A designated in FIG. 2A.

### DETAILED DESCRIPTION OF THE INVENTION

This invention improves upon the existing art by providing a micro-cloth towel in conjunction with a sponge (or other compressible material such as foam) to apply anti-fog solution to a scope to prevent fogging. There are two embodiments, both of which present an outer microcloth surface to clean delicate optics, but with alternative constructions to absorb anti-fog agent applied to the micro-cloth towel.

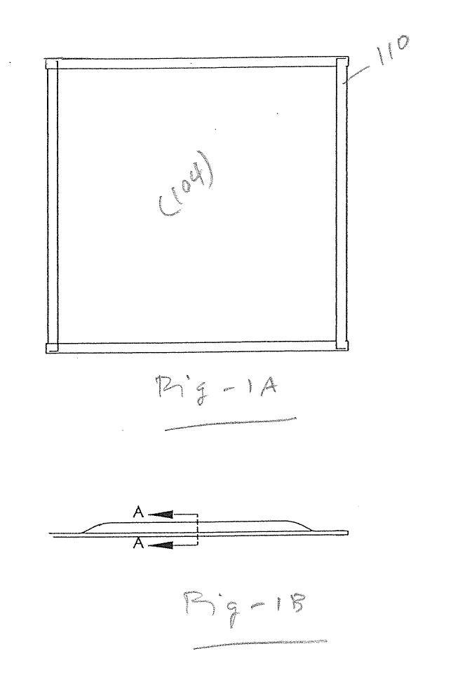

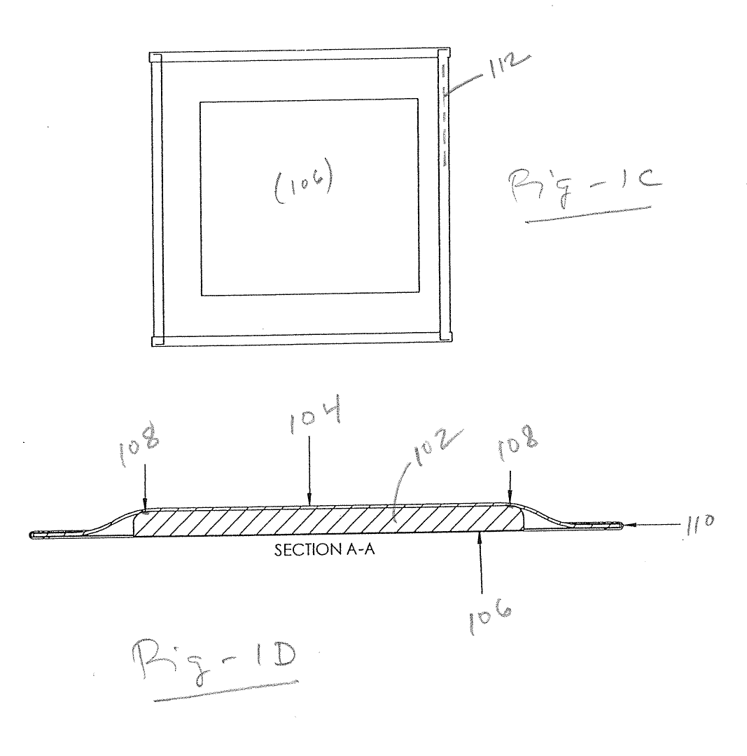

FIGS. 1A-1D depict a first embodiment of the invention that uses only one of a few layers of microcloth 104. Since microcloth alone is not that absorbent, a hydrophilic foam or sponge pad 102 is sandwiched between the microcloth and a lower layer of material 106 (which may or may not be microcloth). A double-sided foam tape may be used as the foam layer, which serves for attachment as well as an impact absorption component.

The upper layer is preferably tacked to the pad 102 at a plurality of glue points 108. The bottom surface of the lower layer of material may include an adhesive for temporarily bonding to a drape, patient skin or other surface. The adhesive would be exposed through the removal of release layer (not shown). The peripheral edge of the article preferably includes an overlap at 110 with a radiopaque thread 112 to prevent loss in a body cavity.

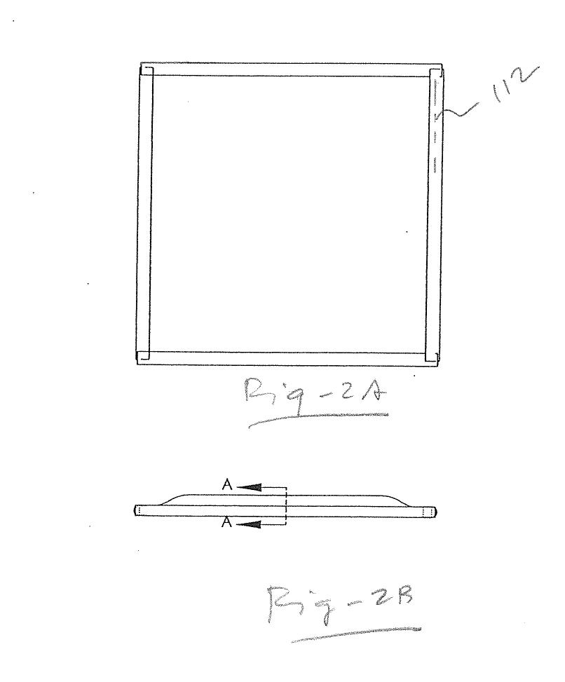

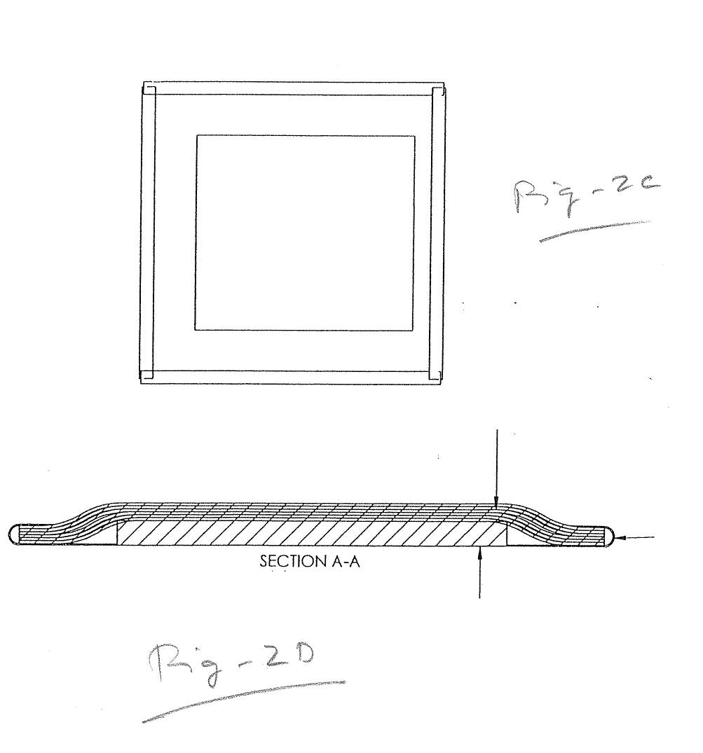

In the second embodiment, shown in FIGS. 2A-2D, uses a plurality of upper material layers to increase absorbance, thereby allowing a hydrophobic (i.e., closed-cell) sponge or foam to be used. A hydrophilic (i.e., open-cell) sponge or foam, including foam tape, is not precluded, however. Six such layers are shown in the Figure, with only the outer layer being micro-cloth. The inner layers may be less expensive absorbent layers such as terrycloth.

In both embodiments, a radiopaque thread 112 is incorporated partly or entirely around the periphery of the article to prevent loss within a patient. The wipe is preferably a square with dimensions on the order of 2 to 4 inches on a side, preferably 3 inches on a side, with a thickness in the range of ⅛ to ½ inches, preferably ¼ inch.

1. A wipe for a laparoscope or other optical device subject to fogging, comprising:
a top outer layer of microfiber cloth;
a bottom outer layer; and
absorbent material disposed between the top and bottom outer layers.

2. The wipe of claim 1, wherein the bottom layer further includes an adhesive layer exposed by a release layer.

3. The wipe of claim 1, wherein the absorbent material is a sponge or foam pad.

4. The wipe of claim 3, wherein the absorbent material is an open-cell foam pad.

5. The wipe of claim 1, wherein the absorbent material includes a plurality of absorbent fabric layers, with at least the outermost layer being a microfiber cloth layer.

6. The wipe of claim 5, including one or more absorbent fabric layers made of terrycloth.

7. The wipe of claim 5, further including a sponge or foam layer between the absorbent fabric layers and the bottom outer layer.

8. The wipe of claim 7, wherein the sponge or foam layer is a closed-cell foam pad.

9. The wipe of claim 1, being a square shape with dimensions on the order of 2 to 4 inches on a side.

10. The wipe of claim 1, having a thickness in the range of ⅛ to ½ inches.

11. The wipe of claim 1, further including a radiopaque thread.
